(19) United States
(12) Patent Application Publication
(10) Pub. No.: US 2004/0176748 A1
(43) Pub. Date: Sep. 9, 2004

(54) Device, method and kit for preventing sexually transmitted disease, generating penile erection and effecting birth control by injecting beneficial agents deep into the male urethra canal prior to sexual intercourse

(76) Inventors: Stanley Abramson, Skokie, IL (US); Brooks Samuel Abramson, Skokie, IL (US)

Correspondence Address: STANLEY ABRAMSON, 4215 GROVE STREET, SKOKIE, IL, 60076, US

(21) Appl. No.: 10/382,316

(22) Filed: Mar. 4, 2003

(51) Int. Cl. A61K 9/22

(52) U.S. Cl. 604/890.1

(57) ABSTRACT

This invention is novel in that it comes in a complete therapeutic kit. It is the only device and method that addresses the problem of STDs and STIs infection and transmission by direct delivery of at least one beneficial <4 pH reducing agent into the penile canal of the urethra and covering all surfaces of the inner canal of the urethra prior to sexual intercourse and it is the only device and method that allows for the option of receiving at least one male penile erection beneficial agent such as PGE-1 that will effect a firm penile erection after adsorption into the base of the penis and the only device and method that allows for the option of receiving at least one beneficial agent such as N-9 or 0-9 that will effect birth control prior to sexual activity along with the STDs and STIs protective and treatment attributes. A device having a flexible tube with a preferred overall length of 140 mm and a inside tube preferred diameter of 0.5 mm and an outside tube preferred diameter of 1.0 mm and preferred curved dome tip to facilitate insertion into the urethra canal from the head of the penis that is attached to a preferred plastic squeeze compressible tube or dose specific squeeze bottle or plunger type hypodermic container having at least one antimicrobial and antiviral beneficial agent and a dispenser tip which allows the tubular part of the device to be attached and delivers at least one beneficial agent through the tubular part of the device into the penile canal.

### BACKGROUND OF THE INVENTION

The advent of new lethal and chronically disabling sexually transmitted infections and diseases combined with the historical STDs and STIs has made it imperative that new techniques be developed that will be attractive to use by non-medically trained individuals and be efficacious to prevent STDs and STIs from spreading during sexual intercourse. The STDs and STIs problems coupled with penile erection dysfunctional problems present a highly unique and desirable opportunity to solve both problems simultaneously. And add the additional related problem of birth control, especially from the male's perspective of having to use condoms, can add a third major feature to this package of penile canal injectable formulas-three potential problems uniquely solved.

The closest known prior art relates to condoms with antispermzoidal coatings, medicated suppository for penal erection, vaginal shields, male potency enhancement oral medications, and vaginal birth control medications.

### BRIEF SUMMARY OF THE INVENTION

The purpose of the invention is to present a method and device coupled with a kit format which will contain all of the individual items required for effecting the method and device thus described for delivering at least one beneficial agent into the penile canal that will coat the interior walls of the male urethra that will result in the prevention of the transmission and subsequent infection of sexually transmitted diseases (STDs) and combined with this beneficial agent(s) can be an additional at least one beneficial agent designed specifically to stimulate male penile erection by being absorbed into the base of the penis and possibly into the blood stream and the lymph system and combined with these two beneficial applications can be an additional at least one beneficial agent designed specifically to act as a birth control agent to kill the male sperm as it exits the male urethra.

The advantages of this invention is that it offers a unique method of STDs and STIs protection coupled with enhanced user pleasure by eliminating the use of a condom or other type of protective shield and/or can be used to deliver a male penile erection beneficial agent simultaneously that may act more quickly than oral medications designed for the same purpose and/or deliver simultaneously a birth control beneficial formulation. All of these applications can be combined in a single dose and/or solely used as separate single application dosages. One step STDs and STIs protection and/or erection enhancement and/or birth control method.

### BRIEF DESCRIPTION OF THE SEVERAL VIEWS OF THE DRAWING

(Not Applicable)

### DETAILED DESCRIPTION OF THE INVENTION

A device, method and kit for preventing sexually transmitted diseases and/or generating and maintaining an erection of the penis for a period of time and/or for birth control. A flexible tubular device that can be inserted a preferred 1.5 inches to 5.0 inches into the genital duct of the male urethra from the head of the penis which enables at least one beneficial agent consisting of a liquid solution, foam or gel composition containing at least one beneficial ingredient such as polysaccharides to maintain the acidity levels and control the pH level in the penile canal which can neutralize sperm and bacteria and inhibit HIV and/or repel HIV-infected cells which can not survive in a high acidity/low pH environment, activated chlorine dioxide and a phosphate compound such as disodium hydrogen phosphate, sodium dihydrogen phosphate, sodium monoflourophosphate, trisodium phosphate and incorporate a formulation that would kill and/or neutralize the male sperms before the sperms leave the interior of the urethra, to be delivered and distributed into the canal and interior walls of the urethra is disclosed for preventing sexually transmitted disease transmission and infection, stimulating and enchancing penile erection and acting as a birth control spermacide formulation. This same device, method and kit solves and/or enhances male penile erection problems by delivering at least one beneficial agent such as prostaglandinE1 PGE 1 (alprostadil) which will effect a firm penile erection after absorption into the base of the penis.

This invention brings an entirely new regimen to the segment of the market that contains men who dislike to use condoms for a variety of reasons which will aid them in incurring/preventing infection and/or transmission of STDs while simultaneously providing them with the option of benefiting from a penile erection agent and an option for a birth control agent contained in the same kit and possibly in the same gel as the anti-STD beneficial agent if they are compatible. If they are not compatible, then the birth control and penile erection beneficial agent (s) can be injected just prior to the anti-STD beneficial agent(s) being injected into the male urethra. Women can effect the same benefits by using the same type of device, method and beneficial agent(s) used for males. This product will also be appealing to women in their relationship with men who insist on not wearing condoms during sexual activities.

The three composite beneficial features of this invention can be used individually as a single purpose remedy, i.e., only as a penile erection stimulation product or only as a STDs and STIs preventative product or only as a birth control product for males. There is no requirement that all three-application features be combined only into a single male penal canal injectable product regimen.

The device consists of a preferred round flexible tube that is up to 100 mm in length with a preferred tapered insertion tip that has a preferred 1.0 mm outside diameter and a preferred 0.5 mm inside diameter and can be attached to a hypodermic type plunger device containing up to 50 ml of beneficial agent(s) or attached to a flexible container containing the desired beneficial agents and being resiliently compressible when subjected to manual pressure or attached to an aerosol configured container which discharges the beneficial agent(s) through the attached tubular pathway directly into the depths of the penal canal.

We claim:

1. A device for preventing sexually transmitted disease and infection, killing male sperm and enhancing penile erection said device comprising:
a) a preferred overall length of up to 140 mm flexible round silicone tube with an outside diameter of 1.0 mm and an inside diameter of 0.5 mm with a preferred curved dome tip to facilitate the insertion of the tube into the urethra canal opening at the head of the penis. And wherein
b) the tubular component described in claim 1.a) can be connected to a delivery system that is a container with a preloaded or re-fillable receptacle containing at least one of the beneficial agent(s) for preventing STDs and STIs transmission, and/or acting as a birth control spermacide, and/or enhancing penile erection. And wherein
c) a pre-loaded or a re-fillable receptacle that can hold up to 50 ml of the beneficial agent(s) that by compression, dose specific squeeze bottle, plunger or pump configuration or aerosol pressure container can deliver and distribute the beneficial agent(s) through the connected tubular part of the device described in claim 1.a. into the penile canal and all inner surfaces of the urethra wall at a depth of up to ten inches. And wherein
d) a device for solving penile erection problems attached to and used in conjunction with claim 1.c) tubular item that contains a dose specific squeeze bottle or tube container having a male penile erection beneficial agent such as a liquid or gel form of sildenafil citrate sold under the name of Viagra™ or prostaglandin E1 PGE1 (alprostadil) sold under the name of Caveject™ which will effect a firm penile erection after adsorption into the base of the penis.

2. A kit for preventing sexually transmitted disease and infection, killing male sperm and enhancing penile erection containing three components of the device described in claim 1. comprising:
a) one or more attachable and disposable sterile flexible silicone tubular attachments as described in claim 1. a);
b) individual containers of each of the three different categories of medication formulas each designed to perform the following distinct functions: 1. prevention of STDs and STIs, 2. Birth control, and 3. enhancing penile erection. The three categories of medication formulas can individually or collectively can be loaded and/or preloaded into the delivery device describe in claim 1b), c) and d); and
c) a pre-loaded or a refillable receptacle that can hold up to 50 ml of the beneficial agent(s) that by compression, plunger or pump configuration or aerosol pressure deliver the beneficial agent(s) through the tubular attachment into the penile canal at a depth of up to ten inches; and
d) a tube of lubricating solution or gel that is applied to the tubular item described in claim 1.a) to ease and facilitate the insertion of the tubular part of the device into the penile canal prior to injecting the beneficial agent of agents into the penile canal.

3. A method for preventing sexually transmitted disease and infection, killing male sperm and enhancing penile erection comprising:
a) the delivery through the tubular part of the device described in claim 1. of at least one beneficial agent into the penile canal derived from pH regulators that will generate normal acidity of 3.8-4.2 that bacteria and virus cannot live in, or detergents and surfactants such as Nonoxynol-9 and Octoxynol-9 used as a spermicide, or anti-retrovirals which interrupt replication of virus or antibiotic peptides which kill bacteria or antibodies which combat infection or microbicides which reduce the transmission of STIs or hydrogen peroxide 3-10% dilution (7% carbamide gel) which will lower the pH environment to <4 which are lethal levels for bacteria and virus;
b) using the device described in claim 1 to deliver, separately or collectively, the preferred efficacious beneficial agent(s) described in claim 3. a) directly into the penile canal which is the easiest and most expeditious and novel method available to prevent sexually transmitted disease and infection, killing male sperm before they leave the penile canal and delivering a penile erection enhancement medication directly and deeply into the base of the penile canal for better absorption and efficacy;
c) using the method described in claim 3.a) and 3.b) the penile canal is protected from in-bound bacteria and virus during intercourse with a STDs or STIs infected sexual partner,
d) using the method described in claim 3.a) and 3.b) the outbound male sperm ejaculated in the semen from the penile canal is killed;
e) the level of the pH of the of at least one beneficial agent described in claim 3.a) is >4 which is ejaculated from the penile canal along with ejaculates and semen which have a pH level >7 into the vagina of a sexual partner lowers the ejaculate and semen pH to a low level more consistent with the pH level in the vagina which is <4 which creates a low pH environment in the vagina which can kill the sperm in the semen; and
f) method described in claim 3. should be used prior to vaginal or anal sexual intercourse.
